(19) United States
(12) Patent Application Publication
(10) Pub. No.: US 2022/0031960 A1
(43) Pub. Date: Feb. 3, 2022

(54) SHIELD TRIGGERED INJECTION DEVICE

(71) Applicant: Novo Nordisk A/S, Bagsvaerd (DK)

(72) Inventor: Martin Johst Christensen, Copenhagen OE (DK)

(21) Appl. No.: 17/275,707

(22) PCT Filed: Sep. 11, 2019
(86) PCT No.: PCT/EP2019/074249

§ 371 Date: Mar. 12, 2021

(30) Foreign Application Priority Data
Sep. 13, 2018 (EP) 18194277.2

(51) Int. Cl. A61M 5/32

(52) U.S. Cl. CPC A61M 5/3272; A61M 5/326; A61M 2005/3267; A61M 2205/581; A61M 2205/582

(57) ABSTRACT

The invention relates to a shield triggered injection device with a triggering mechanism for injecting doses of a liquid drug. The injection device has a housing structure with a distal end and a proximal end, and a needle shield rotationally mounted relatively to the housing structure such that the needle shield is rotatable between a first position and a second position and which the needle shield when rotated from the first position to the second position is guided helically in a helical track. Further, an axially movable trigger element for releasing a set dose to be automatically ejected upon proximal movement of the trigger element against a bias is provided. The trigger element is provided with an axial locking arm which is biased into the helical track to block the passage through the helical track at least in one rotational direction.

### THE TECHNICAL FIELD OF THE INVENTION

The invention relates to a triggering mechanism for an automated shield triggered injection device wherein a set dose is released by moving a trigger element in the proximal direction. The trigger element is preferably moved proximally by an axial movement of a needle shield covering the distal tip of a needle cannula between injections.

### DESCRIPTION OF RELATED ART

Injection devices wherein the needle cannula is hidden by an axially movable needle shield during injection is well known in the patent literature. In one such example provided in WO 2015/062845, the needle shield covering the needle cannula between injections is further provided with a cleaning chamber for cleaning the distal tip of the needle cannula between injections. The cleaning chamber is filled with the same preservative as present in the liquid drug contained in the injection device. In a preferred example, a quantum of the preservative containing liquid drug in the cartridge is filled into the cleaning chamber to operate as the cleaning solvent.

A process of transferring the preservative containing liquid drug from the cartridge and into the cleaning chamber is described in WO 2016/173895. The process involves transforming rotational movement of the protective cap and the needle shield into an axial movement of the cartridge to thereby pump preservative containing liquid drug out from the cartridge and into the cleaning chamber.

As such injection devices are often shield triggered i.e. the set dose is released by axial movement of the needle shield they are often very complex since it must be secured that the cleaning chamber carried by the needle shield is properly filled with preservative containing liquid drug from the cartridge before allowing the user to perform an injection.

Using the same needle shield both as an initiation means for filling the cleaning chamber and for releasing the set dose during injection has however proven to make such injection devices mechanically complicated.

### DESCRIPTION OF THE INVENTION

It is thus an object of the present invention to provide a more simple shield triggered injection device with a needle shield which facilitates both the filling of preservative containing cleaning drug from the cartridge and into the cleaning chamber and allow the use of the same needle shield to release the set dose.

Accordingly, in one aspect of the present invention, a shield triggered injection device with a trigger mechanism is provided. The injection device comprises:
A housing structure with a distal end and a proximal end. The housing structure preferably comprises a plurality of individual parts which are assembled to form the complete housing structure.
A needle shield rotationally mounted relatively to the housing structure to rotate between a first position and a second position. When the needle shield is rotated from the first position to the second position the needle shield is simultaneously guided helically in a helical track.
An axially movable trigger element for releasing a set dose to be automatically ejected upon proximal movement of the trigger element against a bias. The bias preferably arises from a spring delivering an axial force.

By automatically ejected is meant that the injection device comprises a mechanism which is able to eject the set dose without the user delivering the force needed to perform the injection. Such mechanism usually comprises some kind of spring mechanism such that the release of the spring force drives the ejection. An example of such spring mechanism based on a torsion spring is provided in International patent application No.: PCT/EP2018/066236.

The axially movable trigger element is further provided with an axially extending locking arm which is biased into the helical track to block the passage through the helical track at least in one rotational direction.

During rotation from the first position to the second position the needle shield is guided in guiding track and the locking arm is preferably formed such that this rotation is allowed. However, when the needle shield is in the second position, the locking arm is preferably made such that the needle shield is prevented from being rotated back from the second position to the first position.

It is thus possible to divide the rotation of the needle shield into two different kinds of rotational movements which are executed sequentially. A first rotational movement in one rotational direction and which movement is irreversible and which first movement is immediately followed by a second rotational movement which is possible in both rotational directions.

By this separation of the rotational movement it is thus possible to first execute an initiation of the injection device which is irreversible and simultaneously thereafter move the needle shield into a position from which it can be unlocked and locked an unlimited number of times.

The bias on the trigger element and thus on the axially extending locking arm is an axial force operating in the distal direction and is preferably delivered by a resilient member such as a spring. Such spring could be a combined torsion spring and compression such that the spring both delivers a torsional force and an axial force.

In order to guide the needle shield in the helical track, the needle shield is preferably provided with one or more outwardly pointing protrusions. One or more of these protrusions are carried on one or more uprights connected to the needle shield.

The first position is downstream from the axial locking arm and the second position is upstream from the axial locking arm and the one or more outwardly pointing protrusions are movable in the helical track from the first position to the second position such that the protrusions together with the uprights on the needle shield carrying the protrusions passes by the axial locking arm when moving from the first position to the second position.

Once the protrusions on the uprights has passed the locking arm moving from the first position to the second position the axial locking arm prevents any helical movement of the needle shield in the opposite direction. The movement from the first position to the second position is thus irreversible.

The engagement between one or more outwardly pointing protrusions on the upright and the axial locking arm provide a tactile and/or audible signal once the one or more outwardly pointing protrusions passes the axial locking arm. It is thus possible to thereby inform the user of the injection device that the irreversible point has been passed and that the behaviour of the injection device coupled to this first movement has been concluded. In one example this behaviour could be an initiation of the injection device during which initiation the back part of a needle cannula is inserted into the cartridge e.g. followed by a filling of liquid drug from the cartridge and into the cleaning chamber.

In one preferred example the housing structure comprises three different parts; a base part, an initiation part and a cartridge holder part. These three parts are preferably click-fitted together to form one complete housing structure.

The helical track for guiding the needle shield is preferably provided in the housing structure and preferably between the initiator part and the cartridge holder part.

In order to ensure a strictly axial movement of the trigger element it is guided axially in an axial guide track. In one example this axial guide track is a part of the housing structure and preferably of the cartridge holder part. Further, the trigger element is provided a radial protrusion which is guided in the guide track.

The engagement between the one or more outwardly pointing protrusions and/or the upright and the radial protrusion provide a tactile and/or audible signal once the outwardly pointing protrusion passes the radial protrusion such that the user is informed when the protrusions on the needle shield passes the radial protrusion on the trigger element i.e. when the NPR has been concluded and the injection device is unlocked and ready to perform an injection.

The present invention further includes a torsion spring driven injection device comprising the above described triggering mechanism. The configuration of such torsion spring driven injection device is provided in International patent application No.: PCT/EP2018/066236 which is thus included by reference and further depicted in FIG. 4 of the figures.

### Definitions

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel but could also be made from a polymeric material or a glass material. The needle cannula can be anchored in a needle hub or directly in the injection device without the use of a needle hub. If the needle cannula is anchored in a needle hub this needle hub can be either permanently or releasable coupled to the injection device.

As used herein, the term “drug” is meant to encompass any drug-containing flowable medicine capable of being passed through a delivery means such as a hollow needle cannula in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

The term “preservative containing liquid drug” is preferably used to describe a liquid drug containing any kind of a preservative. Such liquid drug could in one example be a blood sugar regulating liquid drug such as insulin, insulin analogue, GLP-1 or GLP-2, and the preservative contained in the liquid drug could in one example be phenol, meta-cresol or any combination thereof. However any kind of preservative can under this term be combined with any kind of liquid drug.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

“Cleaning chamber” is in the present description broadly meant to be any kind of reservoir containing a cleaning solvent to clean at least the distal tip of the needle cannula between subsequent injections. Such cleaning chamber is preferably both distally and proximally sealed by a pierceable septum or the like. However, the proximal septum could be replaced by any kind of sealing which would seal against the outer surface of the needle cannula e.g. a movable plunger with some kind of sealing. The distal septum and the proximal septum or seal of the cleaning chamber defines a confinement containing the cleaning solvent which cleaning solvent in a preferred embodiment is identical to the preservatives contained in the liquid drug used in the specific injection device. In a most preferred solution, the same preservative containing liquid drug is present in both the cleaning chamber and in the cartridge of the injection device thereby avoiding contamination of the preservative containing drug inside the cartridge.

By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses i.e. a force is present in the spring all though the dose size has not been set (the scale drum is at “zero”). Alternatively, the spring can be fully preloaded by the manufacturer with a preload sufficient to empty the entire drug cartridge though a number of doses. Typically, the user activates a latch mechanism provided either on the surface of the housing or at the proximal end of the injection device to release—fully or partially—the force accumulated in the spring when carrying out the injection.

The term “Permanently connected” or “permanently embedded” as used in this description is intended to mean that the parts, which in this application is embodied as a cartridge permanently embedded in the housing, requires the use of tools in order to be separated and should the parts be separated it would permanently damage at least one of the parts.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a perspective view of the injection device with the protective cap mounted.

FIG. 2 show a perspective view of the injection device with the protective cap removed.

FIG. 3 show an exploded view of the injection device according to the invention.

FIG. 4 show a cross sectional view of the spring engine contained in the base part of the housing structure.

FIG. 5 show a perspective view of the trigger element.

FIG. 6A-B show a side view of the engagement between the needle shield, the initiator part and the trigger element in the Out-of-Pack state (with the cartridge holder).

FIG. 7A-B show a side view of the engagement between the needle shield, the initiator part and the trigger element during initiation (with the cartridge holder part visually removed).

FIG. 8 show a side view of the engagement between the needle shield, the initiator part and the trigger element during initiation (with the cartridge holder part visually removed).

FIG. 9A show a side view of the engagement between the needle shield, the initiator part and the trigger element following initiation (with the cartridge holder visually removed).

FIG. 9B show a side view of the engagement between the needle shield, the initiator part and the trigger element following initiation (with the cartridge holder).

FIG. 10A-B show a side view of the engagement between the needle shield, the initiator part and the trigger element when performing NPR (with the cartridge holder).

FIG. 11A-B show a side view of the engagement between the needle shield, the initiator part and the trigger element after NPR has been performed (with the cartridge holder).

FIG. 12A-B show a side view of the engagement between the needle shield, the initiator part and the trigger element during injection (with the cartridge holder).

FIG. 12C show a side view of the engagement between the needle shield, the initiator part and the trigger element during injection (with the cartridge holder part visually removed).

The figures are schematic and simplified for clarity, and they just show details, which are essential to the understanding of the invention, while other details are left out. Throughout, the same reference numerals are used for identical or corresponding parts.

### Detailed Description of Embodiment

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

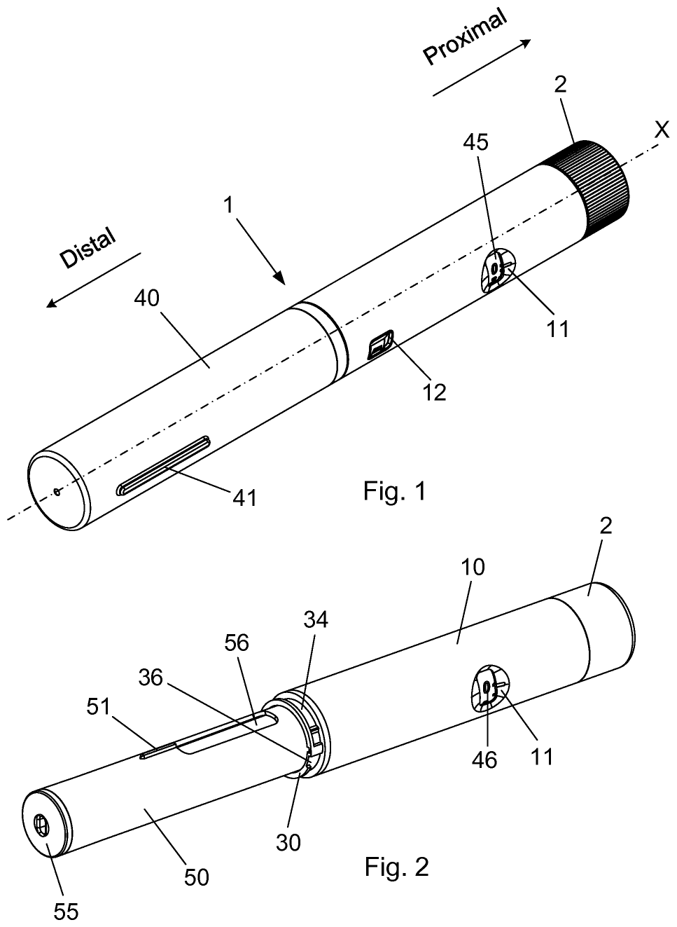

In that context it may be convenient to define that the term “distal end” in the appended figures is meant to refer to the end of the injection device securing the needle cannula and pointing towards the user during injection, whereas the term “proximal end” is meant to refer to the opposite end usually carrying the dose dial button as depicted in FIG. 1. Distal and proximal is meant to be along an axial orientation extending along the longitudinal axis (X) of the injection device as also disclosed in FIG. 1.

FIG. 1 and FIG. 2 disclose the injection device prior to use. The injection device herein described is based on the injection device disclosed in European patent application No. EP18-177945 by Novo Nordisk A/S which is hereby incorporated by reference.

The example used herein relate to a pen shaped injection device. Since pen shaped injection devices has a circular cross section many of the different protrusions and flanges are provided in plural e.g. in pairs. However, in the following they are occasionally explained in singularity and only provided with one reference number although they appear in plural.

The mechanics of the injection device is encapsulated in a housing structure 1 which proximally carries a rotatable dose setting button 2 which the user can rotate to set the size of the dose to be injected.

The distal part of the housing structure 1 is in FIG. 1 covered by a removable protective cap 40 which the user must remove before performing an injection. This protective cap 40 is on the outside provided with a longitudinal raised tongue 41 to enhance the grip when the user rotates the protective cap 40 in order to remove it.

The housing structure 1 is in the disclosed embodiment made up from three different components which are locked together to form the full housing structure 1. The three components are; the base part 10, a cartridge holder part 20 and an initiator part 30.

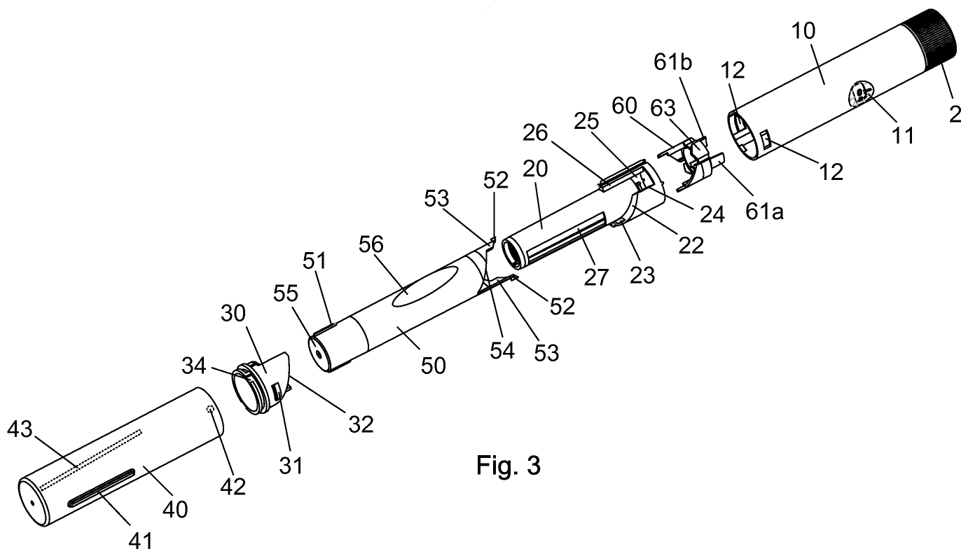

As seen in FIG. 1 and in FIG. 3, the base part 10 is provided with a plurality of openings 12 into which outwardly pointing protrusions 31 provided on the initiator part 30 are clicked such that the base part 10 and the initiator part 30 are both rotational and axially coupled together. There would usually be provided two openings 12 and two protrusions 31 however any number can be used.

The base part 10 is further provided with a window 11 through which a user is able to view indicia 46 printed on the scale drum 45. The indicia 46 usually indicate the dose size set and is usually formed as ciphers or letters printed on the scale drum 45.

FIG. 2 show the injection device with the protective cap 40 removed. The initiator part 30 connecting the base part 10 and the cartridge holder part 20 is on the outer surface provided with a peripheral track 34 having an axial opening 36.

The cartridge holder part 20 which secures the cartridge containing the liquid drug is externally covered by a needle shield 50 which at its distal end holds a cleaning unit 55 for cleaning the distal sharp tip of the needle cannula between injections. The needle shield 50 is able to move in the proximal direction against a bias during injection.

FIG. 3 is an exploded view of the injection device which comprises seen from the left side; the protective cap 40, the initiator part 30, the needle shield 50, the cartridge holder part 20, the triggering element 60 and the base part 10 proximally carrying the dose setting button 2.

The removable cap 40 is on the inner surface provided with one or more inwardly pointing protrusions 42 which engages the peripheral track 34 such that the user is required to rotate the removable cap 40 and the housing structure 1 relatively to each other in order to remove (and to mount) the protective cap 40.

During this mandatory rotation an inwardly pointing tongue 43 inside the protective cap 40 abut a raised bar 51 provided on the rotatable needle shield 50 to thereby transfer rotation from the protective cap 40 to the needle shield 50. The needle shield 50 thus rotates when the protective cap 40 is being removed.

As the inwardly pointing protrusion 42 and the inwardly pointing tongue 43 are provided on the inner surface of the protective cap 40 and pointing inwardly they are not visible from the outside and are consequently illustrated with broken lines in FIG. 3.

The raised bar 51 provided on the needle shield 50 can be placed in any axial position as long as it engages with the inwardly pointing tongue 43 during rotation of the protective cap 40. In FIG. 2 the raised bar 51 is provided adjacent a longitudinal window 56 in the needle shield 50 through which window 56 the user can inspect the content of the cartridge and in FIG. 3 the raised bar 51 is shown in a position at the distal end of the needle shield 50.

In order to properly view the content of the cartridge, the longitudinal window 56 in the needle shield 50 needs to be aligned with a similar window 27 provided in the cartridge holder part 20.

When assembling the injection device, the initiator part 30 slides over the needle shield 50 and over the distal end of the cartridge holder part 20 and clicks to the base part 10 (the outwardly pointing protrusions 31 locks in the openings 12). The trigger element 60 is also placed between the cartridge holder part 20 and the base part 10 during assembly. The initiator part 30 and the cartridge holder part 20 thus fits together such that the initiator part 30, the cartridge holder part 20 and the base part 10 are axially and rotationally locked together to operate as one unitary unit.

The initiator part 30 is proximally provided with a first helical flange 32 and the cartridge holder part 20 has a similar second helical flange 22. When assembled these two helical flanges 22, 32 together form a helical track 35 which guides the outwardly pointing protrusions 52 provided on the needle shield 50.

The outwardly pointing protrusions 52 points radially outwardly from two uprights 53 which connects to a sloped end surface 54 of the needle shield 50. These uprights 53 and indeed the entire needle shield 50 are able to slide under the first helical flange 32, as the outwardly pointing protrusion 52 slides helically in the helical track 35. This is due to the fact that the needle shield 50 slides inside the opening of the initiator part 30. The outwardly pointing protrusions 52 thus have a radial height such that these protrusions 52 are caught and guided by the first helical flange 32.

The second helical flange 22 provided on the cartridge holder part 20 has a similar height as will be explained later, such that the outwardly pointing protrusion 52 is guided by both the first helical flange 32 and by the second helical flange 22.

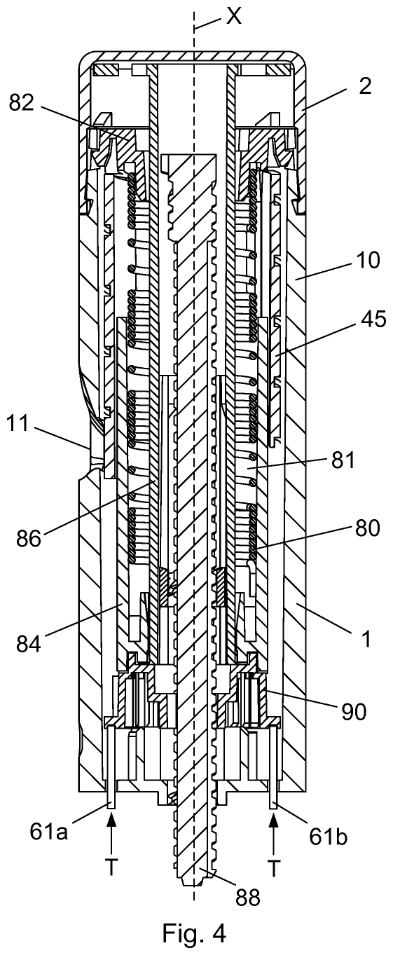

The base part 10 further contains the dose engine which will shortly be explained here. The dose engine is herein disclosed in FIG. 4 which is identical to FIG. 3 in International patent application No.: PCT/EP2018/066236 by Novo Nordisk A/S which is thus incorporated by reference.

The dose engine is a torsion spring mechanism wherein a torsion spring 80 is encompassed between a spring base 82 and a drive element 84. The torsion spring 80 is further provided with a plurality of open windings 81 such that the torsion spring 80 is able to deliver an axial force in addition to the torsional force.

The spring base 82 is anchored to the base part 10 of the housing structure 1 and the drive element 84 is secured to a dose setting tube 86.

Rotation of the dose setting button 2 is transferred to a rotation of the dose setting tube 86 which further rotates the drive element 84 to thereby strain the torsion spring 80.

The drive element 84 is also rotational connected to the scale drum 45 which is guided helically by a groove engagement with the base part 10. During rotation, both when setting a dose and when injecting the set dose, indicia 46 provided on the scale drum 45 rotate helically pass the window 11 in the base part 10.

The piston rod 88 has a longitudinal groove which is engaged by the piston rod driver 90 such that the piston rod 88 and the piston rod driver 90 rotate together. The piston rod 88 is further provided with an external helical thread which is threaded to the housing structure 1.

During dose setting, this piston rod driver 90 is locked to the base part 10 and thus kept in-rotatable such that rotation of the drive element 84 (by rotation of the dose setting tube 86) strains the torsion spring 80 and build up a torque in the torsion spring 80.

In order to release the torque in the torsion spring 80, the piston rod driver 90 is moved in the proximal direction and out of engagement with the base part 10 as disclose in FIG. 4. In this released position, the torque of the torsion spring 80 will rotate the drive element 84, the piston rod driver 90, the dose setting tube 86 and the scale drum 45.

The rotation of the piston rod driver 90 is transferred to a similar rotation of the piston rod 88 which is thus screwed in the distal direction to thereby expel the set dose.

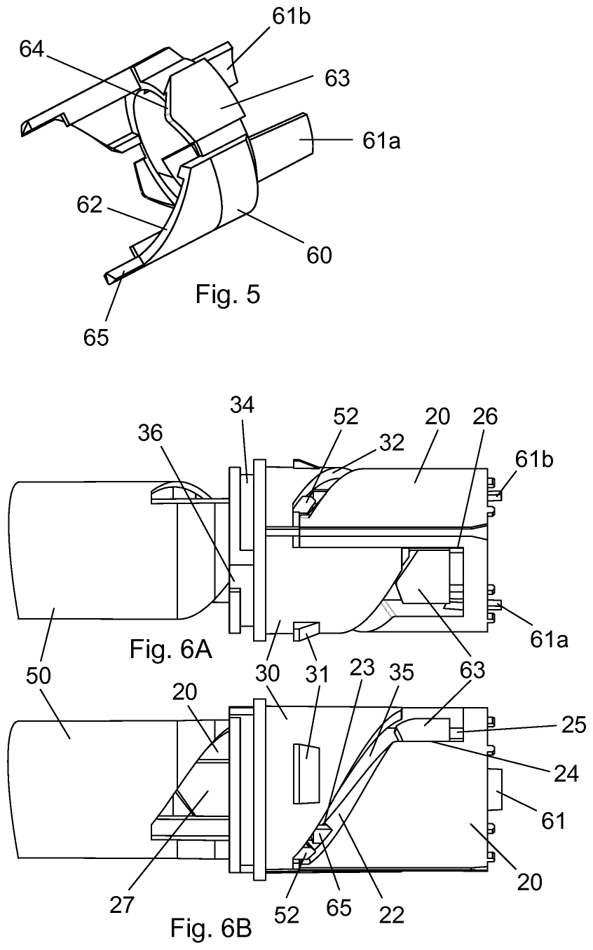

The release element 60 which transfers an axial movement of the needle shield 50 to an axial movement of the piston rod driver 90 is further disclosed in FIG. 5. In the embodiment disclosed in FIG. 5, the release element 60 has two proximally pointing arms 61a,b which abut the piston rod driver 90 when the needle shield 50 and thus the release element 60 is moved proximally during injection. This is indicated by the arrows “T” in FIG. 4. Also in FIG. 4 it is seen that the open windings 81 of the torsion spring 80 apply an axial force onto the piston rod driver 90 and thus onto the release element 60. The release element 60 is thereby movable in the proximal direction against the bias of the compression part of the torsion spring 80. Alternatively two separate springs can be provided: one torsion spring for driving the injection and one ordinary compression spring applying the axial force onto the piston rod driver 90. The release element 60 would thus be movable in the proximal direction against the bias of this compression spring.

The release element 60 is further provided with two helical flanges 62 which each terminate in an axial locking arm 65. Further, two radial protrusions 63 are symmetrically provided, the use of which will be explained later.

As best seen in FIG. 3 and in FIG. 6A-B, the trigger element 60 is mounted inside the cartridge holder part 20 such that the axial locking arms 65 extend through openings 23 in the second helical flange 22 and into the helical track 35 and the two helical flanges 62 on the release element 60 abut against the proximal backside of the second helical flange 22 on the cartridge holder part 20 when the trigger element 60 is biased in the distal direction by the compression part of the torsion spring 80. Further, the engagement between the radial protrusion 63 and the guide opening 25 provided in the cartridge holder part 20 secures that the trigger element 60 can only move axially in relation to the cartridge holder part 20.

The guide opening 25 is bordered by a first longitudinal wall 24 and a second longitudinal wall 26 which abut the side surfaces of the radial protrusion 63 to guide the radial protrusion 63 axially. As best seen in FIG. 3 and in FIG. 6A-B, the second helical flange 22 terminates into the first longitudinal wall 24 and the second longitudinal wall 26 abut against a similar longitudinal wall provided on the initiator part 30. Further, the height of the radial protrusion 63 is such that the radial protrusion 63 is able to slide under the cartridge holder part 20 at the proximal end of this.

However, in order to release a set dose, the trigger element 60 needs to be moved in the proximal direction by the needle shield 50 as will be explained.

FIG. 6 A-B show side views of the front end on the injection device in the out-of-pack state. In FIG. 6B the injection device has been rotated 90° around the X-axis relatively to FIG. 6A.

Out-of-Pack

The protective cap 40 has visually been removed in FIG. 6A and FIG. 6B all though the injection device in the Out-of-Pack state usually is delivered with the protective cap 40 mounted. The outwardly pointing protrusions 52 on the needle shield 50 is located in the start of the helical track 35 formed between the first helical flange 32 on the initiator part 30 and the second helical flange 22 provided on the cartridge holder part 20. The most distal part of the needle shield 50 has also been visually cut away in FIG. 6 and in the following figures.

When the injection device is in the out-of-pack state, it is not possible to move the needle shield 50 axially since the protrusion 52 would thus abut the first helical flange 22 which would prevent the needle shield 50 from moving in the proximal direction. It is thus not possible to release a dose before the needle shield 50 and the protrusions 52 has been rotated to an unlocked position in which the protrusions 52 is allowed to move proximally.

Further, in the Out-of-Pack state the presence of the protective cap 40 hinders access to the needle shield 50 as e.g. illustrated in FIG. 1. Also in the out-of-pack state, the inwardly pointing protrusion 42 is parked in the bottom of the peripheral track 34 opposite the axial opening 36.

Initiation

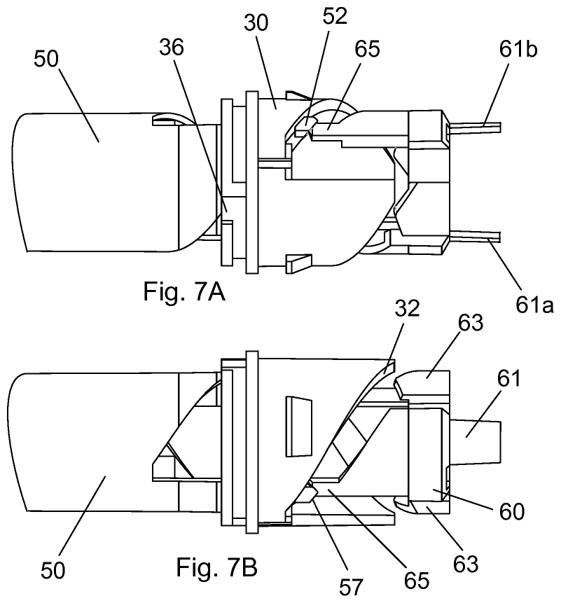

In order to initiate the injection device, the user needs to rotate the needle shield 50. This is automatically done when the user rotates the protective cap 40 to remove it. FIG. 7A-B illustrates the situation when the user starts to rotate the protective cap 40 which rotation is transferred to a similar rotation of the needle shield 50 due to the engagement between the inwardly pointing tongue 43 and the raised bar 51.

For a better visual showing, both the protective cap 40 and the cartridge holder part 20 have been cut away in FIG. 7A-B.

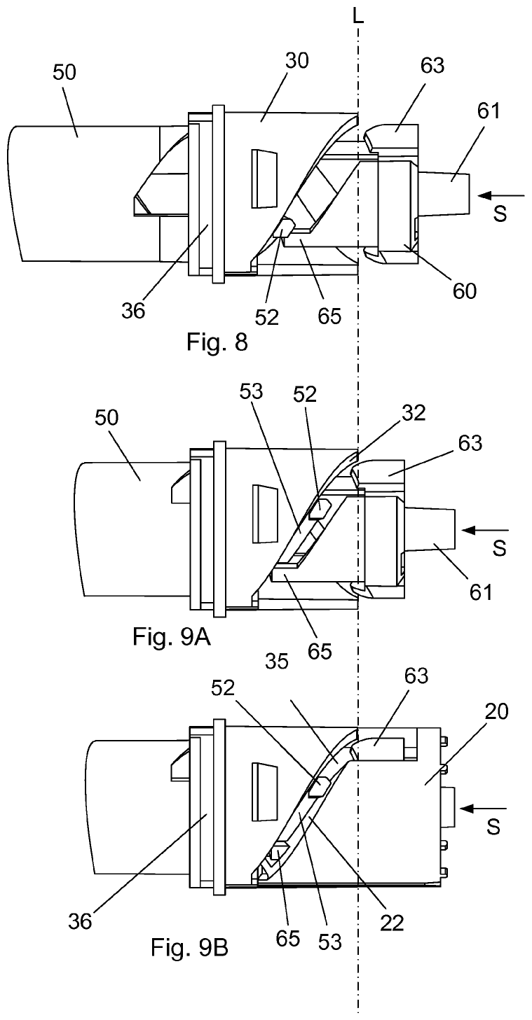

When the V-shaped proximal end 57 of the protrusion 52 engages with the distal end of the axial locking arm 65, the axial locking arm 65 and thus the trigger element 60 are moved slightly in the proximal direction against the bias of the compression part of the torsion spring 80. However, this axial movement is not sufficient to move the piston rod driver 90 out of its engagement with the base part 10 to thereby release the toque stored in the torsion spring 80, but the axial movement of the trigger element 60 allows the outwardly pointing protrusion 52 to move pass the axial locking arm 65 and into the position disclosed in FIG. 8.

The proximal movement of the trigger element 60 during initiation is further illustrated by the line “L” in FIG. 8 and in FIG. 9A-B. It is thus seen that the trigger element 60 moves slightly in the proximal direction in FIG. 8 as the outwardly pointing protrusion 52 passes by the axial locking arm 65. Once the outwardly pointing protrusion 52 together with the upright 53 has passed the axial locking arm 65 as illustrated in FIG. 9A-B, the bias (arrow “S”) of the compression part of the torsion spring 80 moves the trigger element 60 distally back into its initial position wherein it hinders the upright 53 with the protrusion 52 from being moved backwards in the helical track 35.

During initiation, the needle hub carrying the needle cannula is moved proximally such that the needle cannula penetrates into the cartridge. Further, the needle hub forces the cartridge a short distance in the proximal direction such that liquid drug is pumped from the interior of the cartridge and into the cleaning chamber as explained in European patent application No. EP18-177945 by Novo Nordisk A/S.

In summary, once the outwardly pointing protrusion 52 together with the upright 53 has moved pass the axial locking arm 65 as disclosed in FIG. 9A-B, the compression part of the torsion spring 80 moves the trigger element 60 and thus the axial locking arm 65 back into its initial position which is accompanied by a distinct tactile and audible signal informing the user that the initiation is finished. The compression force of the torsion spring 80 is indicated by the arrow “S” in FIG. 9A-B. In this position, the axial locking arm 65 hinders that the upright 53 with the outwardly pointing protrusion 52 can be rotated back in the clockwise direction. Consequently once the initiation is done it cannot be undone.

In FIG. 9A, the cartridge holder part 20 has been visually removed to further show the details. When the upright 53 has passed the axial locking arm 65 it is thus not possible to rotate back the outwardly pointing protrusion 52 and henceforth the needle shield 50. Further it is not possible to move the outwardly pointing protrusion 52 and thus the needle shield 50 in the axial direction as the guiding track 35 prevents movement of the outwardly pointing protrusion 52 in a purely axial direction.

Once the upright 53 has passed the axial locking arm 65, the initiation is done and the needle cannula has been attached to the cartridge which has also been moved slightly in the proximal direction to build up a pressure inside the cartridge which pressure pumps liquid drug from the cartridge and into the chamber in the cleaning unit 55. At the same time, the inwardly pointing protrusion 42 inside the protective cap 40 has reached the axial opening 36 in the initiator part 30 such that the user can now remove the protective cap 40 by simply pulling it axially off.

NPR

Once the initiation of the injection device is done, the user needs to perform the NPR (Needle Pressure Relief). This is done by further rotating the needle shield 50 such that the distal end of the needle shield 50 carrying the cleaning unit 55 is moved to a position wherein the distal tip of the needle cannula is located just outside the cleaning chamber to thereby release the pressure in the lumen of the needle cannula and inside the cartridge.

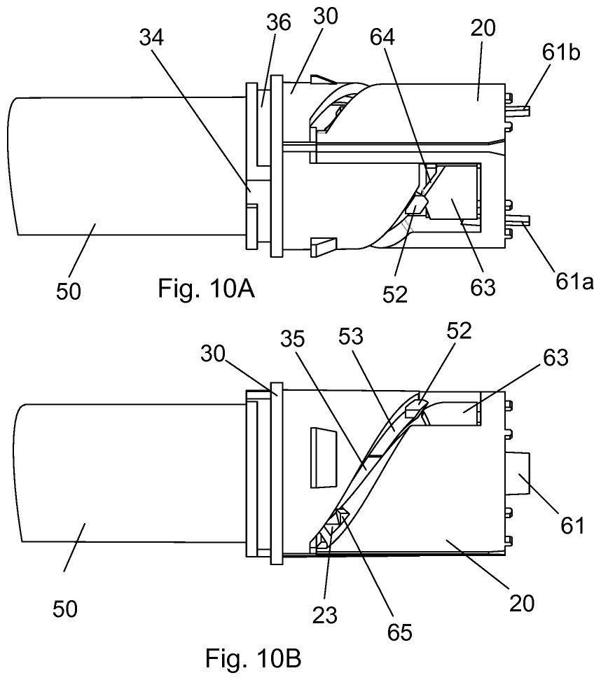

As seen in FIG. 10A-B, the protrusion 52 and the upright 53 engages the distal V-shaped end 64 of the radial protrusion 63. This engagement moves the radial protrusion 63 and thus the trigger element 60 slightly in the proximal direction against the bias of the compression part of the torsion spring 80.

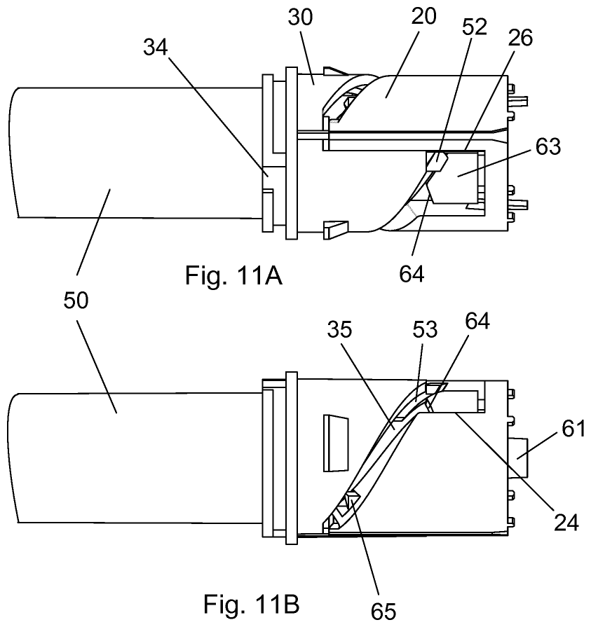

Once the outwardly pointing protrusion 52 has passed the radial protrusion 63 as disclosed in FIG. 11A-B, the bias of the compression part of the torsion spring 80 moves the trigger element 60 and the radial protrusion 63 back into its initial position which provides the user with a tactile and audible signal indicating that the NPR state has now been concluded and that the injection device is now in the injection state.

In this position, the outwardly pointing protrusion 52 together with the upright 53 abut against the second longitudinal wall 26 such that the outwardly pointing protrusion 52 and the upright 53 follow along the second longitudinal wall 26 into the guide opening 25 when the trigger element 60 is moved axially during injection as further explained. The injection device is henceforth unlocked and ready to inject.

Injection

Once the NPR is done the outwardly pointing protrusion 52 and the upright 53 abuts the radial protrusion 63 and is aligned with the guide opening 25 by abutment with the second longitudinal wall 26 as illustrated in FIG. 11A-B which means that the needle shield 50 and the trigger element 60 can now move freely in the proximal direction.

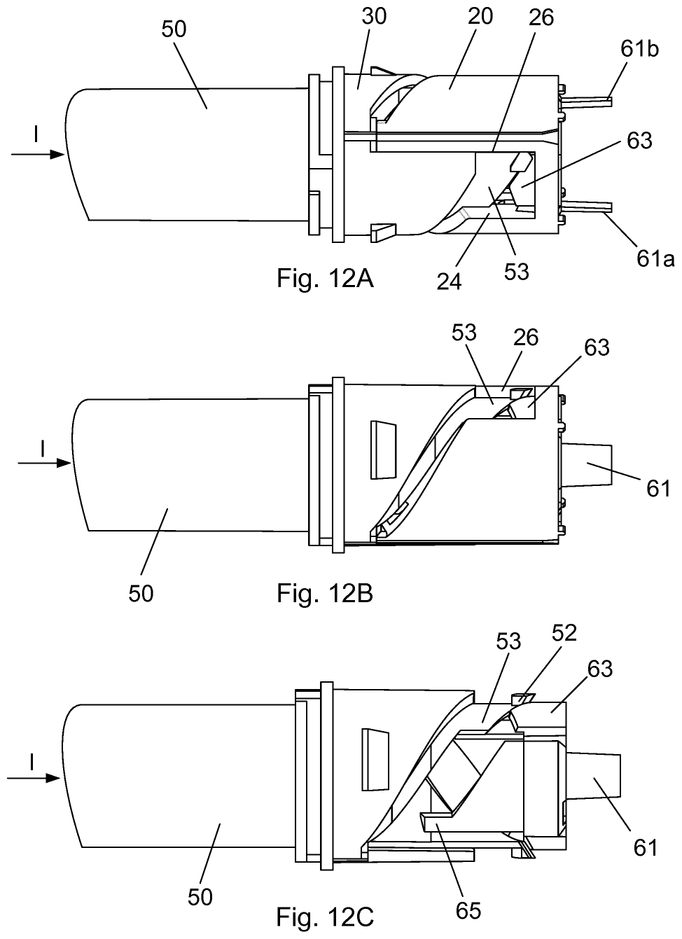

An injection is thus performed by pushing the needle shield 50 against the skin of the user as illustrated by the arrow “I” in FIG. 12A-C. The proximal movement of the needle shield 50 moves the trigger element 60 in the proximal direction as well. When the trigger element 60 moves proximally, the proximally pointing arms 61a,b pushes the piston rod driver 90 out of engagement with the base part 10 (as illustrated in FIG. 4) which thus allows the torsion spring 80 to rotate the piston rod driver 90 and thus screw the piston rod 88 forward in its threaded engagement with the housing structure 1.

Following an injection, the bias of the compression part of the torsion spring 80 moves the trigger element 60 back into the NPR position disclosed in FIG. 11A-B and from this position, the user can rotate the needle shield 50 out of the NPR position by manually rotating the needle shield 50 in the clockwise direction until the upright 53 again abut the axial locking arm 65 as disclosed in FIG. 9A-B. In this position, the distal tip of the needle cannula is brought back into the cleaning chamber of the cleaning unit 55 and the needle shield 50 is locked form axial movement.

Some preferred embodiments have been shown in the foregoing, but it should be stressed that the invention is not limited to these, but may be embodied in other ways within the subject matter defined in the following claims.

1. A shield triggered injection device with a triggering mechanism, comprising:
a housing structure having a distal end and a proximal end,
a needle shield which is rotationally mounted relatively to the housing structure to rotate between a first position and a second position and wherein the needle shield when rotated from the first position to the second position is guided helically in a helical track,
an axially movable trigger element for releasing a set dose to be automatically ejected upon proximal movement of the trigger element against a bias,
wherein the trigger element is provided with an axial locking arm which is biased into the helical track to block the passage through the helical track at least in one rotational direction.

2. The shield triggered injection device with a triggering mechanism according to claim 1, wherein the bias is an axial force operating in the distal direction and delivered by a resilient member such as a spring.

3. The shield triggered injection device with a triggering mechanism according to claim 1, wherein the needle shield is provided with one or more outwardly pointing protrusions.

4. The shield triggered injection device with a triggering mechanism according to claim 3, wherein the one or more outwardly pointing protrusions are guided in the helical track.

5. The shield triggered injection device with a triggering mechanism according to claim 3, wherein the one or more outwardly pointing protrusions are carried on one or more uprights connected to the needle shield.

6. The shield triggered injection device with a triggering mechanism according to claim 1, wherein the first position is downstream from the axial locking arm and the second position is upstream from the axial locking arm.

7. The shield triggered injection device with a triggering mechanism according to claim 3, wherein the one or more outwardly pointing protrusions are movable in the helical track from the first position to the second position

8. The shield triggered injection device with a triggering mechanism according to claim 3, wherein the axial locking arm prevents movement of the one or more outwardly pointing protrusion from the second position and back into the first position.

9. The shield triggered injection device with a triggering mechanism according to claim 3, wherein the engagement between one or more outwardly pointing protrusions and/or the upright and the axial locking arm provide a tactile and/or audible signal once the one or more outwardly pointing protrusions passes the axial locking arm.

10. The shield triggered injection device with a triggering mechanism according to claim 1, wherein the housing structure comprises a base part, an initiation part and a cartridge holder part.

11. The shield triggered injection device with a triggering mechanism according to claim 10, wherein the helical track is provided between the initiator part and the cartridge holder part.

12. The shield triggered injection device with a triggering mechanism according to claim 1, wherein the trigger element is guided axially in a guide track.

13. The shield triggered injection device with a triggering mechanism according to claim 12, wherein the trigger element is provided with a radial protrusion guided in the guide track.

14. The shield triggered injection device with a triggering mechanism according to claim 13, wherein the engagement between the one or more outwardly pointing protrusions and/or the upright and the radial protrusion provide a tactile and/or audible signal once the outwardly pointing protrusion passes the radial protrusion.

15. The shield triggered injection device with a triggering mechanism according to claim 1 comprising a torsion spring drive mechanism.
